(19) United States
(12) Patent Application Publication
(10) Pub. No.: US 2021/0369740 A1
(43) Pub. Date: Dec. 2, 2021

(54) Synthesis of Anti-inflammatory and Anti-cancer Agents through Fungal Transformation of Mibolerone

(71) Applicants: Muhammad Iqbal Choudhary, Karachi (PK); Atia-tul- Wahab, Karachi (PK); Mahwish Siddiqui, Karachi (PK); Almas Jabeen, Karachi (PK); Nimra Naveed Shaikh, Karachi (PK); Atta-ur- Rahman, Karachi (PK)

(72) Inventors: Muhammad Iqbal Choudhary, Karachi (PK); Atia-tul- Wahab, Karachi (PK); Mahwish Siddiqui, Karachi (PK); Almas Jabeen, Karachi (PK); Nimra Naveed Shaikh, Karachi (PK); Atta-ur- Rahman, Karachi (PK)

(21) Appl. No.: 17/400,164

(22) Filed: Aug. 12, 2021

(51) Int. Cl. A61K 31/58

(52) U.S. Cl. CPC A61K 31/58

(57) ABSTRACT

Four new analogues, 17β-hydroxy-7α,17α-dimethylestr-4,6-diene-3-one (2), 11β,17β-dihydroxy-7α, 17α-dimethyl-estra-1,3,5-triene-3-one (3), 3α,10β,17β-trihydroxy-7α,17α-dimethyl-5α-estrane (4), and 17β-hydroxy-7α,17α-dimethyl-5α-estrane-3,6-dione (5) of anabolic drug mibolerone (1) were synthesized. Derivatives 2 (IC₅₀=3.83 ±0.3 μM) and 3 (IC₅₀=4.24 ±0.2 μM) were identified as potent anti-inflammatory agents against T-cell proliferation. Derivative 4 (IC₅₀=28.5 ±0.07 μM) showed a potent anti-inflammatory activity against TNF-α production. In addition, compounds 1 (IC₅₀=46.0 ±2.4 μM), 2 (IC₅₀=54.4 ±0.3 μM), 3 (IC₅₀=49.1 ±0.4 μM), 4 (IC₅₀=58.0 ±0.1 μM) and 5 (IC₅₀=52.7 ±0.3 μM) showed a remarkable anti-inflammatory activity against NO^{⋅} production. Metabolite 4 (IC₅₀=0.072 ±0.001 μM) showed a potent inhibitory activity against human placental aromatase. Compound 1 (IC₅₀=24.19 ±2.1 μg/mL) was found to be cytotoxic against BJ normal cell line, while metabolites 2-5 were identified as non-cytotoxic.

### BACKGROUND OF THE INVENTION

Inflammation is an inherent process, which directs the immune cells to the site of lesions/injuries in order to eradicate infections, activate healing process, and repair damaged tissues. During the process of inflammation activated leukocytes and, plasma proteins participate to produce immune response. However, if inflammation persist for longer time it become chronic. This chronic inflammation is a response of long-term exposures to injurious stimuli that destroy tissues. The cytokine TNF (tumor necrosis factor) is a major regulator of inflammatory responses and initially known for its anti-tumor activity. It is produced by macrophages, T-cells and Natural Killer (NK) cells, which regulate the growth and differentiation of many cell types and responsible for various cellular signaling events leading to necrosis or apoptosis. However, the increased production of TNF-α contributes in the remodeling of tissues and, known to be involved in the pathogenesis of autoimmune and inflammatory diseases. NO (nitric oxide) free radical, produced from 1-arginine, and at physiological concentrations have important role in the regulation of various cellular events and immune response. The overproduction NO is also involved in the pathogenesis of numerous diseases, such as cancers, Alzheimer's, cardiovascular, and diabetes. The dysregulation of immune system is the basis of various autoimmune and chronic inflammatory diseases and associated with over production of inflammatory cytokines like TNF-α, IL-1β, oxidative species including NO as well as abnormal proliferation of T-cells so targeting these molecules by novel compounds could result in better therapeutic outcomes [Siddiqui et al., J. Adv. Rev. 2020, 24, 69; Moro-Garcia, et al., Front. Immunol. 2018, 9, 339; Ibrahim et al., Steroids, 2020, 162, 108694; Sakai and Kobayashi 2015. Pathol. 65, 344-354; Burger and Dayer, Arthritis Res. Ther. 2002, 4, 1-8].

Breast cancer is one of the deadliest diseases in females globally accounting for one-third of all cancers. Generally, breast cancers arise from sequential mutations in the body due to genetic instability and/or environmental factors. They are characterized by the production of abnormal cells in the breast with higher proliferation rates, which often survive against available chemotherapeutic agents. Around 80% of all breast cancers are estrogen-responsive, and therefore treated by inhibiting the aromatase enzyme. Aromatase enzyme catalyzes the synthesis of estrogen hormone. Estrogens play important roles in the development of female organs, and regulation of reproductive system. They also serve as signaling molecules for cell proliferation. Aromatase inhibitors reduce the production of estrogens, and therefore use for the treatment of disorders due to the imbalance between estrogen and androgen levels [Ghuge et al., Curr. Enzym. Inhib. 2020, 16, 45; Waks and Winer, JAMA, 2019, 321, 288; Sun et al., Int. J. Biol. Sci. 2017, 13, 1387; Chumsri et al., J. Steroid Biochem. Mol. Biol. 2011, 125, 13; Altundag and Ibrahim, The Oncologist, 2006, 11, 553].

Steroids are among the most important bioactive natural or semi-synthetic organic compounds due to their anabolic, anti-androgenic, anti-cancer, anti-inflammatory, anti-microbial, anti-aromatase, and contraceptive properties [Siddiqui et al., J. Adv. Rev. 2020, 24, 69; Atia-tul-Wahab et al., Bioorg. Chem. 2018, 77, 152; Maltais et al., J. Comb. Chem. 2004, 6, 443]. Mibolerone (1) is a potent anabolic-androgenic steroid, sold under the trade name of Cheque Drops by the Upjohn Company, USA. It is an orally active synthetic derivative of 17α-alkylated nandrolone. Compound 1 is also used for the treatment of estrus (oestrus) in the female dogs [Siddiqui et al., PloS One, 2017, e0171476; Meyers-Wallen, Theriogenology, 2007, 68, 1205].

Microbial transformation is a robust technique to derivatize steroidal drugs into stereo-, regio-, and chemo-selective analogues without using any toxic and expensive reagents. This technique is effectively applied to bring structural modifications in almost all classes of organic compounds, where synthetic methodologies are difficult [Siddiqui et al., J. Adv. Rev. 2020, 24, 69; Atia-tul-Wahab et al., Bioorg. Chem. 2018, 77, 152; Choudhary et al., Front. Pharmacol. 2017, 8, 900; Bianchini et al., Front. Microbiol. 2015, 6, 1433].

### BRIEF SUMMARY OF THE INVENTION

In continuation of our biotransformation studies on bioactive steroids [Siddiqui et al., Phytochem. Lett. 2021, 44, 147; Chegaing et al., Steroids, 2020, 162, 108679; Siddiqui et al., J. Adv. Rev. 2020, 24, 69; Ibrahim et al., Steroids, 2020, 162, 108694; Hussain et al., RSC Advances, 2020, 10, 451; Atia-tul-Wahab et al., Bioorg. Chem. 2018, 77, 152; Choudhary et al., Front. Pharmacol. 2017, 8, 900; Siddiqui et al., PloS One, 2017, e0171476; Bano et al., Steroids, 2016, 112, 168], we have focused on an anabolic steroidal drug, mibolerone (1).

Biotransformation of anabolic drug mibolerone (1) with Aspergillus niger yielded four new derivatives, 17β-hydroxy-7α,17α-dimethylestr-4,6-diene-3-one (2), 11β,17β-dihydroxy-7α,17α-dimethyl-estra-1,3,5-triene-3-one (3), 3α,10β,17β-trihydroxy-7α,17α-dimethyl-5α-estrane (4), and 17β-hydroxy-7α,17α-dimethyl-5α-estrane-3,6-dione (5).

Derivatives 2 (IC₅₀=3.83 ±0.3 μM) and 3 (IC₅₀=4.24 ±0.2 μM) showed potent anti-inflammatory activity against T-cell proliferation, in comparison to substrate 1 (IC₅₀=6.55 ±0.6 and standard drug prednisolone (IC₅₀=9.75 ±0.03 μM), while metabolites 4 and 5 were found to be inactive in vitro. Interestingly, metabolite 4 (IC₅₀=28.5 ±0.07 μM) was identified as a potent anti-inflammatory agent against TNF-α production, when compared with substrate 1 (IC₅₀=115.8 ±0.7 and standard pentoxifylline (IC₅₀=341.0 ±2.1 μM). Whereas, compounds 2, 3, 5, and 6 were identified as inactive to TNF-α production in vitro. In addition, compounds 1 (IC₅₀=46.0 ±2.4 μM), 2 (IC₅₀=54.4 ±0.3 μM), 3 (IC₅₀=49.1 ±0.4 μM), 4 (IC₅₀=58.0 ±0.1 μM) and 5 (IC₅₀=52.7 ±0.3 μM) showed a remarkable anti-inflammatory activity against nitric oxide (NO) production, as compared to standard inhibitor, L-NMMA (NG-Monomethyl-L-arginine, monoacetate salt) (IC₅₀=128.2 ±0.8 μM) in vitro.

Metabolite 4 (IC₅₀=0.072 ±0.001 μM) showed a potent inhibitory activity against human placental aromatase enzyme, in contrast to compounds 1 (IC₅₀=0.372 ±0.009 μM), 2 (IC₅₀=0.31 ±0.028 μM), 3 (IC₅₀=0.317 ±0.012 μM), and standard drug exemestane (IC₅₀=0.232 ±0.031 μM) while compound 5 was found to be inactive in vitro.

Substrate 1 (IC₅₀=80.0 ±2.1 μM) was found to be cytotoxic against BJ normal cell line (human fibroblast), while metabolites 2-5 were identified as non-cytotoxic in vitro.

### BREIF DESCRIPTION OF THE DRAWINGS

FIG. 1 depicts the structures of mibolerone (1), and its new metabolites 2 and 3 synthesized via Aspergillus niger-mediated transformation of drug 1, along with their anti-inflammatory, and anti-cancer activities, and cytotoxicity against human fibroblast (BJ) cell line.

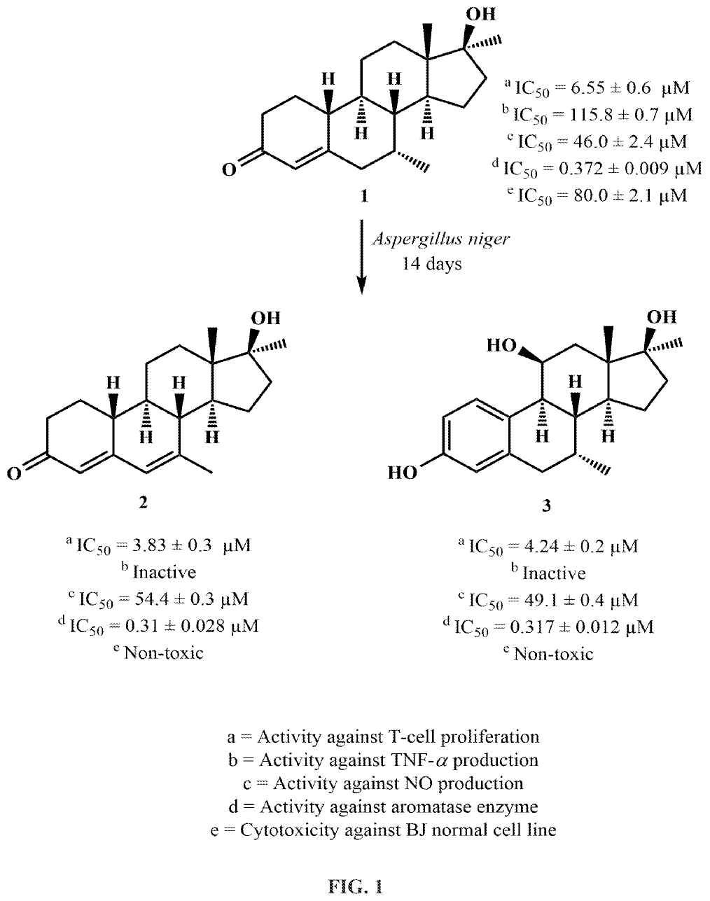

FIG. 2 depicts the structures of mibolerone (1), and its new metabolites 4 and 5 synthesized via Aspergillus niger-mediated transformation of drug 1, along with their anti-inflammatory, and anti-cancer activities, and cytotoxicity against human fibroblast (BJ) cell line.

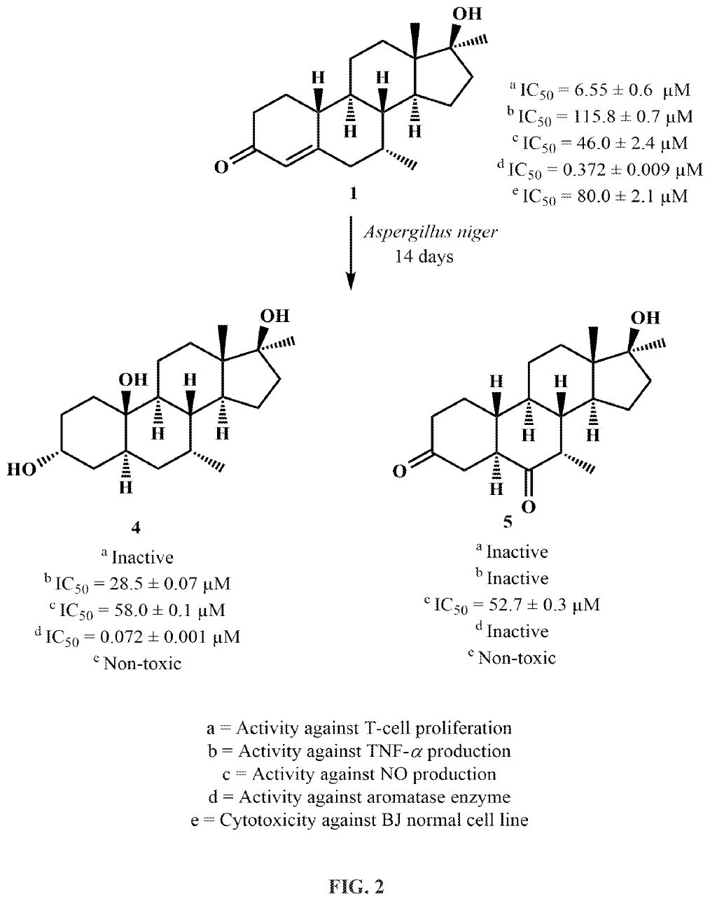

### DETAILED DESCRIPTION OF THE INVENTION

### Experimental

Drug: Mibolerone (1) was procured from Shenzhen Simeiquan Biotechnology Company Limited, China.

Fungi: Aspergillus niger (ATCC 10549) was purchased from American Type Culture Collection (ATCC).

### Media Preparation

10 L media for the growth of Aspergillus niger was prepared by mixing 100 g glucose, 50 g NaCl, 50 g peptone, 50 g KH₂PO₄, and 100 mL glycerol in 10 L distilled water.

### Fermentation

Based on small-scale screening results, 10 L of media was prepared by mixing aforementioned ingredients. Media (400 mL) was transferred into 25 Erlenmeyer flasks of 1000 mL, cotton plugged, and autoclaved at 121° C. Media was cooled at room temperature, and inoculated with A. niger culture under sterilized conditions. A. niger containing flasks were placed for four days on a rotary shaker (121 rpm). After the mature growth of A. niger culture in each flask, 3 g of mibolerone (1) was dissolved in methanol (25 mL), and dispensed (1 mL) in each flask. These flasks were again placed on rotary shaker (121 rpm) at 25° C. for fourteen days.

### Extraction

After incubation, EtOAc (ethyl acetate) was added in each flask to stop the reaction, and filtered to separate fungal masses. Resulting filtrate was extracted thrice with 30 L of EtOAc. Anhydrous Na₂SO₄ was added in the extract to absorb moisture, filtered, and evaporated by using rotary evaporator.

### Isolation and Purification

The resulting gummy crude material was subjected to column chromatography (CC) for fractionation by using a mobile phase of hexanes-acetone (with 5-100% gradients of acetone). Four main fractions were obtained. Compounds 2 (CH₃CN—H₂O; 6/4, R_{T}=25 min), 3 (CH₃CN—H₂O; 6/4, R_{T}=21 min), 4 (H₂OACN, 3/7; Rₜ =27 min), and 5 (CH₃CN—H₂O; 7/3, R_{T}=24 min) were purified through recycling RP-HPLC from fractions 1 to 4, respectively.

### 17β-Hydroxy-7α,17α-dimethylestr-4,6-diene-3-one (2)

White solid; melting point: 154-153° C.; UV λₘₐₓ (log ε): 298 (6.4); [α]_{D}²⁵=+67.3 (c 0.001, CH₃OH); IR (CHCl₃) υₘₐₓ (cm⁻¹): 3430 (OH), 1655 (α, β-unsaturated C═O), 1612 (C═C); EI-MS m/z (rel. int., %): 300.2 [M⁺] (37), 282.3 (38), 267.2 (36), 229.2 (23), 174.1 (27), 147.2 (26), 136.1 (100); HREI-MS m/z: 300.2090 [M⁺] (C₂₀H₂₈O₂, calcd. 300.2089); ¹H-NMR (δ) (500 MHz; CD₃OD) H₂-1 (1.56, overlap; 1.33, m), H₂-2 (2.38, overlap, 2[H]), H-4 (5.63, s), H-6 (6.06, s), H-8 (2.37, overlap), H-9 (1.57, overlap), H-10 (2.33, overlap), H₂-11 (2.02, m; 1.65, overlap), H₂-12 (1.89, m; 1.68, overlap), H-14 (1.15, overlap), H₂-15 (1.83, m; 1.39, m), H₂-16 (2.32, overlap; 1.47, m), H₃-18 (0.95, s), H₃-19 (0.96, s), H₃-20 (1.19, s); ¹³C-NMR (δ) (125 MHz; CD₃OD) C-1 (32.0), C-2 (38.4), C-3 (203.3), C-4 (121.7), C-5 (157.7), C-6 (127.8), C-7 (163.4), C-8 (42.4), C-9 (48.0), C-10 (47.2), C-11 (28.0), C-12 (38.9), C-13 (48.0), C-14 (47.4), C-15 (26.3), C-16 (28.3), C-17 (80.9), C-18 (14.6), C-19 (25.9), C-20 (25.7).

### 11β,17β-Dihydroxy-7α,17α-dimethyl-estra-1,3,5-triene-3-one (3)

White solid; yield (%): 4.5 mg (0.32); melting point: 213-215° C.; UV λₘₐₓ (log ε): 230 (5.90); [α]_{D}²⁵=+54.3 (c 0.001, CH₃OH); IR (CHCl₃) υₘₐₓ (cm⁻¹): 3322 (OH), 1609, 1500 (aromatic C═C); EI-MS m/z (rel. int., %): 316.2 [M⁺] (51.4), 240.2 (88), 227.1 (100), 185.1 (17), 172.1 (52), 145.1 (44), 83.0 (57); HREI-MS m/z: 316.2053 [M⁺] (C₂₀H₂₈O₃, calcd. 316.2038); ¹H-NMR (δ) (500 MHz; CD₃OD) H-1 (7.14, d; J_{1,2}=8.5 Hz), H-2 (6.59, dd; J_{1,2}=8.5 Hz; J_{2,4} =2.5 Hz), H-4 (6.47, d; J_{1,2} =2.5 Hz), H₂-6 (3.01, dd; J_{6,6} =16.1 Hz; J_{6,7} =4.6 Hz), H-7 (2.04, overlap), H-8 (2.03, overlap), H-9 (2.43, overlap), H-11 (4.79, d; J_{1,2} =2.9 Hz), H₂-12 (1.88, overlap; 1.65, overlap), H-14 (1.59, overlap), H₂-15 (11.58, overlap, 2[H]), H₂-16 (2.44, overlap, 2[H]), H₃-18 (0.95, s), H₃-19 (0.85, d; J_{19,7} =6.9 Hz), H₃-20 (1.23, s); ¹³C-NMR (δ) (125 MHz; CD₃OD) C-1 (128.0), C-2 (114.2), C-3 (156.0), C-4 (117.1), C-5 (139.0), C-6 (39.6), C-7 (29.0), C-8 (37.7), C-9 (44.5), C-10 (128.1), C-11 (69.4), C-12 (39.2), C-13 (47.0), C-14 (48.8), C-15 (23.2), C-16 (39.2), C-17 (82.6), C-18 (17.3), C-19 (12.5), C-20 (26.5).

### 3α,10β,17β-Trihydroxy-7α,17α-dimethyl-5α-estrane (4)

White solid; melting point: 194-196° C.; [α]_{D}²⁵=+63.3 (c 0.002, CH₃OH); IR (KBr) υₘₐₓ (cm⁻¹): 3446 (O—H), 2938 (C—H), 1055 (C—O); EI-MS m/z (rel. int., %): 322.2 [M⁺] (13), 304.2 (36), 264.2 (33), 233.2 (42), 215.1 (100), 135.1 (32); HREI-MS m/z: 322.2509 [M⁺] (C₂₀H₃₄O₃, calcd. 322.2508); ¹-NMR (δ) (500 MHz; CD₃OD) H₂-1 (1.76, m; 1.66, overlap), H₂-2 (1.60, overlap; 1.46, m), H-3 (3.92, br. s), H₂-4 (1.91, m; 1.62, overlap), H-5 (1.57, overlap), H₂-6 (2.29, m; 1.10, m), H-7 (1.80, m), H-9 (1.99, overlap), H₂-11 (1.55, overlap; 1.27, overlap), H₂-12 (1.51, m; 1.28, overlap), H-14 (1.40, m), H₂-15 (1.56, m, 2[H]), H₂-16 (1.82, overlap, 2[H]), H₃-18 (0.88, s), H₃-19 (0.97, d, J_{19,7}=6.9 Hz), H₃-20 (1.18, s); ¹³C-NMR (δ) (125 MHz; CD₃OD) C-1 (32.6), C-2 (30.4), C-3 (66.7), C-4 (39.1), C-5 (35.1), C-6 (34.8), C-7 (30.1), C-8 (38.7), C-9 (38.9), C-10 (74.7), C-11 (24.2), C-12 (32.6), C-13 (46.6), C-14 (47.2), C-15 (21.2), C-16 (39.4), C-17 (82.3), C-18 (14.3), C-19 (17.8), C-20 (26.1).

### 17β-Hydroxy-7α,17α-dimethyl-5α-estrane-3,6-dione (5)

White solid; UV λₘₐₓ (log ε): 209 nm (5.8); melting point: 161-162° C.; [α]_{D}²⁵=47.6 (c 0.001); IR υₘₐₓ (cm⁻¹): 3450 (OH), 1709 (C═O); EI-MS m/z (rel. int., %): 318.2 [M⁺] (100), 300.2 (18), 261.2 (17), 229.2 (25), 177.1 (21), 121.1 (11), 95.1 (09); HREI-MS m/z: 318.2198 [M⁺] (calc. 318.2195) (C₂₀H₂₈O₂); ¹H-NMR (δ) (600 MHz; CDCl₃) H₂-1 (1.52, overlap; 1.32, overlap), H₂-2 (2.39, overlap; 2.25, m), H₂-4 (2.42, overlapped, 2[H]), H-5 (2.67, m), H-8 (1.70, overlap), H-9 (1.43, overlap), H-10 (1.53, overlap), H₂-11 (1.94, m; 1.26, overlap), H₂-12 (1.79, m, 2[H]), H-14 (1.54, overlap), H₂-15 (1.51, overlap; 1.24, overlap), H₂-16 (2.36, m; 1.43, overlap), H₃-18 (0.86, s), H₃-19 (1.07, d, J_{19,7}=7.3 Hz), H₃-20 (1.23, s); ¹³C-NMR (δ) (150 MHz; CDCl₃) C-1 (31.0), C-2 (40.4), C-3 (211.1), C-4 (40.6), C-5 (49.1), C-6 (212.5), C-7 (46.5), C-8 (39.5), C-9 (48.0), C-10 (45.0) C-11 (25.0), C-12 (38.6), C-13 (45.9), C-14 (44.7), C-15 (22.1), C-16 (31.0), C-17 (81.3), C-18 (13.6.3), C-19 (11.4), C-20 (25.9).

### T-Lymphocyte Proliferation Assay

T-Lymphocytes were isolated from human heparinized blood. In this assay, blood (10 mL) from healthy human volunteer was aseptically collected, and mixed with incomplete RPMI-1640 (10 mL). The mixture was layered on LSM (lymphocyte separation medium) (5 mL), and tubes were centrifuged at 400 g for 20 min at 25° C. The resulting buffy layer was mixed with incomplete RPMI-1640, and centrifuged at 300 g for 10 min at 4° C. The PBMCs containing pellet was resuspended in FBS (5%) containing RPMI (1 mL). Proliferation of T-cells was conducted by Alamar blue assay. The isolated PMBCs (2×10⁶ cells/mL) were plated in a round bottom 96-well tissue culture plates. Phytohemagglutinin-L (7.5 μg/mL) was used to activate peripheral blood T-cells. Three different concentrations of compounds in triplicates were added. The plates were reincubated for 48 h at 37° C. in CO₂ (5%). A one-tenth volume of Alamar blue dye was added, incubated for 4 h. Absorbance was read in spectrophotometer at wavelengths of 570, and 600 nm.

### Cytokine Inhibition Assay

In this assay, cells (THP-1) from the ECCC (UK) were grown, and maintained in the RPMI-1640, comprising mercaptoethanol (50 μM), 10% FBS (fetal bovine serum), L-glutamine (2 Mm), glucose (5.5 Mm), sodium pyruvate (1 mM), and HEPES (10 Mm). At confluency of 70%, 2.5×10⁵ cells/mL were added in a 24-well plates of culture. PMA (phorbol myristate acetate) (20 ng/mL) was added to differentiate them into the macrophage mimicking cells, followed by incubating at 37° C. for 24 hours in the presence of 5% CO₂. Lipopolysaccharide B of Escherichia coli (50 ng/mL) was used to stimulate the cells, treated with test samples, and placed for 4 hours in 5% CO₂ at 37° C. The level of TNF-α in supernatants was determined on ELISA by using human Duo Set kit (R & D Systems, Minneapolis, USA), as per instructions of the manufacturer.

### Nitric Oxide Assay

The mouse macrophage cell line J774.2 (ECACC) was cultured in 75 cc flasks IWAKI in DMEM, supplemented with fetal bovine serum GIBCO (10%) and streptomycin/penicillin (1%). Flasks were placed in atmosphere of humidified air containing 5% CO₂ at 37° C. Cells were seeded in 96-well plate (10⁶ cells/mL), and were induced by E. coli lipopolysaccharide (LPS) (30 μg/mL). The test compounds were added at three different concentrations (1, 10 and 100 μM). The plate was incubated in 5% CO₂ at 37° C. Nitrite accumulation in culture supernatant was measured by using Griess method.

### Human Placental Aromatase Inhibition Assay Protocol

Transformation of testosterone to 17β-estradiol can be used for the measurement of aromatase enzyme activity. The activity of aromatase enzyme was determined in a reaction mixture (1 mL), containing 2 mg/mL aromatase enzyme, 10 μM testosterone, 0.1 M potassium phosphate buffer at pH 7.4, and 10 μL of test compound (0.1 mM). Mixture was pre-incubated for 10 minutes at 37° C. NADPH (1 mM) was added in the mixture, and incubated for 20 minutes. Trichloroacetic acid (10%, w/v) (100 μL) was added to stop the reaction, centrifuged at 12,000 g for 10 minutes, pellet was discarded, and the supernatant (17β-estradiol) was extracted with N-butyl chloride (1 mL). The extract (17β-estradiol) was dried, and the product quantity was determined by UPLC (column ACE Generix 5 μm C₁₈150×4.6 mm) using isocratic elution of the mobile phase containing triethylamine (0.1%) in ACN/H₂O (45:55, v/v), and pH 3.0 (adjusted by orthophosphoric acid) with a flow rate of 1.2 mL/min at 200 nm. Calculations were performed by following formula:

$$\% Inhibition = 100 - \frac{( Peak area of test sample )}{( Peak area of control )} \times 100$$

### Results and Discussion

Metabolite 2 displayed the [M⁺] in the HREI-MS at m/z 300.2090 (C₂₀H₂₈O₂, calcd. 300.2089), suggesting dehydrogenation of substrate 1 (m/z 302.2, C₂₀H₃₀O₂). Dehydrogenation in drug 1 was further determined through IR, 1D-, and 2D-NMR spectroscopic techniques. Thus the structure of new derivative 2 was deduced as 17β-hydroxy-7α,17α-dimethylestr-4,6-diene-3-one.

The HREI-MS of derivative 3 showed the [M⁺] at m/z 316.2053 (C₂₀H₂₈O₃, calcd. 316.2038), suggesting dehydrogenation and oxidation of substrate 1 (m/z 302.2, C₂₀H₃₀O₂). Aromatization, and hydroxylation in metabolite 3 was determined through the IR, 1D-, and 2D-NMR spectroscopic techniques. The structure of new metabolite 3 was identified as 11β,17β-dihydroxy-7α,17α-dimethyl-estra-1,3,5-triene-3-one.

Derivative 4 displayed its [M⁺] in the HREI-MS at m/z 322.2509 (C₂₀H₃₄O₃, calcd. 322.2508), suggesting addition of an oxygen and four hydrogen atoms in substrate 1 (m/z 302.2, C₂₀H₃₀O₂). These structural changes in drug 1 were further determined through IR, 1D-, and 2D-NMR spectroscopic techniques. Thus the structure of new transformed product 4 was deduced as 3α,10β,17,β-trihydroxy-7α,17α-dimethyl-5α-estrane.

The HREI-MS of metabolite 5 showed the [M⁺] at m/z 318.2198 (C₂₀H₃₀O₃) (calc. 318.2195), suggesting addition of an oxygen atom in the compound 1 (m/z 302.2) (C₂₀H₃₀O₂). The structure of compound was determined as 17β-hydroxy-7α,17α-dimethyl-5α-estrane-3,6-dione (5) through the IR, 1D-, and 2D-NMR spectroscopic techniques.

Changes in the structure of mibolerone (1) have affected their anti-inflammatory, and anti-cancer activities. Dehydrogenation between C-6/C-7 in derivative 2 has increased its activity against T-cell proliferation, and decreased against NO production, as compared to substrate 1. Aromatization in ring A, along with β hydroxylation at C-11 in derivative 3 have also increased its activity against T-cell proliferation, and decreased against NO production, when compared with substrate 1. Moreover, structural changes in derivatives 2, and 3 have diminished their activity against TNF-α production. Reduction of CO group at C-3 into a OH, reduction between C-4/C-5, and β hydroxylation at C-10 in derivative 4 have increased its activity against TNF-α production and aromatase enzyme, and diminished against T-cell proliferation. Reduction between C-4/C-5, and presence of CO group at C-6 in derivative 5 have diminished its activity against T-cell proliferation, TNF-α production, and aromatase enzyme, in comparison to substrate 1.

What is claimed is:

1. A method of treatment of chronic inflammations, comprising on administration of an effective amount of newly developed anti-inflammatory agents having formulae 2-5 or their isomers, salts or solvates, or co-crystals in suitable pharmaceutical excipients, adjuvant, carrier, or diluent to humans, and animals in need thereof.

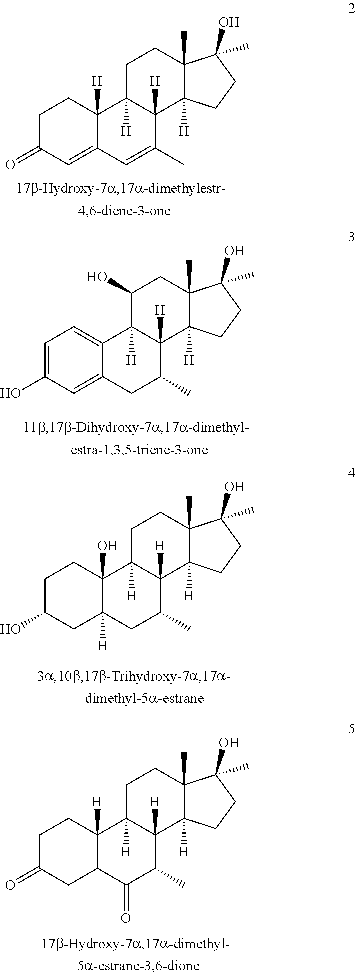

2. Formulae 2 and 3, as in claim 1, showed a potent anti-inflammatory activity against T-cell proliferation with the IC₅₀ values of 3.83 ±0.3 μM, and 4.24 ±0.2 μM, respectively, in comparison to standard drug prednisolone (IC₅₀=9.75 ±0.03 μM) in vitro.

3. Formula 4, as in claim 1, showed a potent anti-inflammatory activity against TNF-α production with the IC₅₀ value of 28.5 ±0.07 μM, when compared with standard pentoxifylline (IC₅₀=341.0 ±2.1 μM) in vitro.

4. Formulae 2, 3, 4, and 5, as in claim 1, showed a remarkable anti-inflammatory activity against nitric oxide (NO^{⋅}) production with the IC₅₀ values of 54.4 ±0.3 μM, 49.1 ±0.4 μM, 58.0 ±0.1 and 52.7 ±0.3 respectively, as compared to standard L-NMMA (IC₅₀=128.2 ±0.8 μM).

5. Formulae 2-5, as in claim 1, can be synthesized by biotransformation of anabolic drug mibolerone (1) or through the chemical synthesis.

6. A method of treatment of diseases associated with the over-expression of aromatase enzyme, including breast cancers, and male infertility, based on administration of effective amount of newly developed aromatase inhibitors having formulae 2-4 or their isomers, salts or solvates, or co-crystals in suitable pharmaceutical excipients, adjuvant, carrier, or diluent to humans, and animals in need thereof.

7. Formulae 2, 3, and 4, as in claim 6, showed a potent inhibitory activity against human placental aromatase with the IC₅₀ values of 0.31 ±0.028 0.317 ±0.012 and 0.072 ±0.001 respectively, in contrast to standard exemestane (IC₅₀=0.232 ±0.031 μM) in vitro.
